(19) United States
(12) Patent Application Publication
(10) Pub. No.: US 2006/0041318 A1
(43) Pub. Date: Feb. 23, 2006

(54) Laminar skin-bone fixation transcutaneous implant and method for use thereof

(76) Inventor: Donald T. Shannon, Trabuco Canyon, CA (US)

Correspondence Address: Manfred E. Wolff;Intellepharm, Inc., 1304 Morningside Drive, Laguna Beach, CA, 92651, US

(21) Appl. No.: 10/922,018

(22) Filed: Aug. 19, 2004

(51) Int. Cl. A61F 2/28; A61F 2/78

(52) U.S. Cl. 623/23.46; 623/32

(57) ABSTRACT

A laminar skin-bone fixation transcutaneous implant adapted for implantation in a residual limb of an amputee comprising a biocompatible bone implant post having a first segment adapted for bone implantation, a transcutaneous segment attached to one or more biocompatible porous layers adapted for vascularization and stable sealable ingrowth by skin cells, and a third segment adapted for adapted for attachment to a prosthesis. The implant may include an uppermost biocompatible non-porous elastomer layer having a multiplicity of perforations. Methods for use of the implant and an article of manufacture for its packaging are also taught.

### BACKGROUND ART

The present invention is related to methods and apparatus for transcutaneous implants for prosthetic appliances. More specifically, this invention is related to methods and devices particularly adapted to transcutaneous implants that are effectively designed to be anchored to both bone and skin, whereby the use of such implants is broadly enabled, and wherein the functional utility, ease of use, and wide applicability of such implants in medical practice constitutes progress in science and the useful arts. Furthermore, the present invention teaches processes for the use of the devices of the invention in medical practice, bionics and related allographic research arts.

Transcutaneous implants: A variety of medical conditions require the installation of transcutaneous implant devices in patients. These are devices that penetrate the skin and include prosthetic appliances intended to replace avulsed or amputated limbs or digits. Limb loss can occur due to trauma, infection, diabetes, vascular disease, cancer and other diseases. The causes of congenital limb differences are frequently unknown. In the past, many cases of limb difference were attributed to the use of drugs, such as thalidomide by the mother during pregnancy. The Amputee Coalition of America estimates that there were approximately 1,285,000 persons in the U.S. living with the limb loss (excluding fingers and toes) in 1996. The prevalence rate in 1996 was 4.9 per 1,000 persons. The incidence rate was 46.2 per 100,000 persons with vascular disease, 5.86 per 100,000 persons secondary to trauma, 0.35 per 100,000 secondary to malignancy of a bone or joint. The birth prevalence of congenital limb deficiency in 1996 was 25.64 per 100,000 live births. The prevalence rate is highest among people aged 65 years and older—about 19.4 per 1,000. It is conservatively estimated that the worldwide population for amputees is triple the U.S. number or approximately 3.9 million people. Many of these individuals experience functional limitations of their prosthetic devices.

The successful development of a transcutaneous implant would improve function of lower limb & upper extremity amputees and enable wearing of prosthetic digits by improving proprioception, increasing their range of motion and improving wear time. Although a few attempts have been made to design and produce such devices, it is still the case that relatively few functionally operational successes have been achieved. As recently as 2001 only a single success has been alleged as empirically feasible and documented in developing an osseointegrated above the knee transcutaneous implant, as will be discussed later. This translates into a significant unmet medical need that presents a commercial product development opportunity.

After limb loss, the resulting lower extremity and upper extremity residual limbs are traditionally fitted with custom made rigid or semi-rigid sockets, onto which mechanical prosthetic devices are attached. The suitability and acceptability of a given prosthesis depends in the first instance on the effectiveness of this linkage between the prosthesis and the residual limb. A number of disadvantages arise from the use of socket devices for this purpose. For example, in one 2001 study of transfemoral amputees with a prosthesis, the most frequently reported problems that had led to reduction in quality of life were heat/sweating in the prosthetic socket, sores/skin irritation from the socket, inability to walk in woods and fields, and inability to walk quickly. In general, the problems of socket prostheses include:
Weight-bearing is transmitted from the skeleton through the soft tissues to the encircling socket and movements are exerted via the skin-prosthesis interface.
Skin is not a satisfactory high load bearing structure and often breaks down under load, becoming inflamed and uncomfortable. In severe cases, pressure sores are formed that are difficult to heal.
The socket that receives the residual limb can commonly become sweaty and uncomfortable.
Especially in leg prostheses, the soft tissues of the residual limb are deformed and compressed under load, leading to a rhythmic shape change termed “pumping” when the patient undertakes certain activities such as walking.
Osseoperception—sensory perception by the patient of position and load through osseous receptors—is grossly reduced owing to the absence of direct communication between the prosthesis and the bone.
Where a joint is involved, the external prosthesis is usually moved by muscle groups situated at a distance from the attached prosthesis, thereby producing motion that is inefficient and unnatural.

In order to more precisely address and attempt to ameliorate some or all of these difficulties, the design and construction of a prosthesis with a direct connection to the bone is required. Here, all of the undesirable consequences of load bearing by the soft tissues—inflammation, sweating, discomfort, “pumping”, and inefficient and unnatural motion—are ameliorated. In addition, osseoperception—the transmission of sensory information through the skeleton—is much improved relative to socket prostheses. Yet skeletal fixation of prosthetic limbs requires communication of the implant with both hard and soft tissues, leading to distinct tissue implant interfacial problems associated with both fixation in bone and with soft tissue attachment in the transcutaneous region.

In very general terms, an improved transcutaneous prosthesis necessarily incorporates an intraosseous transcutaneous element for direct attachment to bone as well as a means successfully to connect the prosthesis to the skin of the residual limb. Such a transcutaneous device could be used not only for leg amputees, but also for other medical needs such as arm, finger and thumb amputations, facial epistheses, and anchored external hearing aids. It is necessary to distinguish here between transcutaneous prostheses that penetrate the skin from other prostheses that do not, such as the well-known knee replacements, metacarpophalangeal joint replacements, and interphalangeal joint replacements. Also, I do not consider here dental applications of intraosseous transcutaneous implants.

The challenges posed by a transcutaneous undertaking were already enunciated more than a quarter of a century ago by Winter (Winter, G. D. (1974) Transcutaneous implants: reactions of the skin-implant interface. J Biomed Mater Res, 8, 99-113) who pointed out that the design of the transcutaneous component Is a key element. Thus, a long term implant penetrating the skin presents novel problems of maintaining a permanent hole in the epidermis, and the risks of tissue breakdown and infection have to be overcome. Likewise, artificial devices that penetrate the skin present problems that include infection and scar formation. For example, specially evolved and biologically differentiated structures such as horns, hair, feathers, fingernails, hoofs, teeth, and antlers are examples where nature has solved the problems of “transcutaneous devices”, but duplicating this technology has been fraught with problems, even though attempts were made to do so as far back as 150 years ago. The skin in animals like man and the pig is basically organized in three layers. The epidermis is 25-50μ thick, and is composed wholly of cells and is situated over the dermis. The dermis is 2-3 mm thick and is made up mainly of extracellular fibers. It is the structural layer of the skin that gives it its toughness. The hypodermis, 12 mm or more thick is mostly fat and is the insulating layer. The epidermis prevents ingress of dirt, microorganisms and harmful radiation.

When injured, the dermis and hypodermis are repaired by new formation of fibrous tissue, which originates from loose connective tissue around blood vessels within about 1.5 mm of the periphery of the wound. The epidermis has a continual and relatively rapid turnover throughout life and, if injured, possesses great powers of regeneration by epidermal cell migration from the epidermis at the wound margin. The epidermis is organized as a continuous stratified sheet of cells and normally the cells move from the basement membrane towards the surface, becoming flattened and eventually lifeless squamae of keratin in the process. Normally, the physical contact between epidermal cells suppresses their inherent mobility. When a gap is cut in a sheet of epidermis the cells at the edge are no longer suppressed in this manner and move across the wound surface until they contact homologous cells in another sheet of epidermis and continuity is restored. In the presence of a solid transcutaneous implant such as a suture or a skeletal attachment prosthesis, the cells “burrow down” in a restless attempt to restore epidermal continuity, thereby forming abscesses even at the bone. However, if the epidermal cells encounter an uninjured collagenous matrix, for example the periodontal membrane around teeth that consists of bundles of collagen fibers, they cease this process. Thus, the concept has been developed that the transcutaneous component of a skeletal attachment prosthesis should be sufficiently porous to allow the ingrowth of fibrous tissue.

Beginning in the early 20^{th} century the technique of transcutaneous fixation of fractures was developed but all of the work ended in failure owing to infection of the area surrounding the implant. More recently, the skin interfacing potential of various velours, felts, foams and rough cast surfaces of some polymers was investigated by bonding these substances to solid core silastic rods using Dow-Corning brand of Medical Adhesive Type A. These skin penetrating rods were implanted onto the dorsum of canines, goats, and swine but infections again defeated these and subsequent related efforts.

In man, more successful recent efforts used sintered metal fiber-web materials. Staubach (Staubach, K. H. and Grundei, H. (2001) [The first osseointegrated percutaneous prosthesis anchor for above-knee amputees]. Biomed Tech (Berl), 46, 355-61) drove a surface-structured metal pin capable of supporting large loads into the medullary canal of the thighbone of an above knee amputee. Screwed to the end of the pin was a conical metal adapter attached to a silicone cylinder whose right-angled distal end terminates in a titanium mesh. Wound healing at the metal/tissue interface was complication-free and the patient was able to return to his normal occupation, and has had no further problem for a period of over one-year. After a half-century of attempts, this appears to be the first long-term success in this area. Although these metallic meshes are reported to be superior to polyethylene terephthalates sold under the trademark DACRON® (Walboomers, F., Paquay, Y. C. and Jansen, J. A. (2001) A new titanium fiber mesh-cuffed peritoneal dialysis catheter: evaluation and comparison with a Dacron-cuffed tenckhoff catheter in goats. Perit Dial Int, 21, 254-62) non-metallic fibers have heretofore been considered to offer advantages to metal fibers with regard to porosity and maintenance of structural integrity under conditions of flexing. The failure modes of percutaneous devices were reviewed two decades ago (von Recum, A. F. (1984) Applications and failure modes of percutaneous devices: a review. J Biomed Mater Res, 18, 323-36). Prominent among them are mechanically induced failure, infection, and marsupialization, in which epidermal cells burrow under the implant and convert it from a percutaneous to an extracutaneous status. In general, infection resulting from failure of the skin interface with transfemoral transcutaneous prosthetic devices has blocked their successful application and only very few successes, notably that of Staubach as already mentioned, have been recorded. A recent Patent Application Publication (Blunn, G., Cobb, J., Goodship, A. and Unwin, P. (2003) Transcutaneous Prosthesis. U.S. Patent Application Publication U.S. 2003/0171825) purports to describe a transcutaneous prosthesis but gives no hint regarding how the von Recum failure modes would be avoided.

Thus, in spite of extended efforts in academic medicine and the pharmaceutical industry, there remains a major unmet medical need for improvement in the construction and function of devices particularly adapted to a laminar skin-bone fixation transcutaneous implant adapted for stable sealable ingrowth by skin cells and for implantation in a residual limb of an amputee. Even though prostheses are used extensively in medical practice, prior devices, products, or methods available to medical practitioners have not adequately addressed the need for bone implants adapted for vascularization (Brauker, J. H., Carr-Brendel, V. E., Martinson, L. A., Crudele, J., Johnston, W. D. and Johnson, R. C. (1995) Neovascularization of synthetic membranes directed by membrane microarchitecture. J Biomed Mater Res, 29, 1517-24) and stable sealable ingrowth by skin cells. Thus, as pioneers and innovators attempt to provide new methods and apparatus particularly adapted to skin-bone fixation transcutaneous residual limb implants, my invention of laminar skin-bone fixation transcutaneous residual limb implants that include a transcutaneous segment attached to one or more biocompatible porous layers adapted for vascularization and stable sealable ingrowth by skin cells provide improved implant procedures that are broadly enabled. The functional utility, ease of use, and wide applicability of the device of my invention in medical practice will make it safer, more universally used, and of higher quality than any other. No other device has approached these objectives in combination with simplicity and reliability of operation, until the teachings of the present invention. It is respectfully submitted that other references merely define the state of the art or show the type of systems that have been used to alternately address those issues ameliorated by the teachings of the present invention. Accordingly, further discussions of these references has been omitted at this time due to the fact that they are readily distinguishable from the instant teachings to one of skill in the art.

### OBJECTS AND SUMMARY OF THE INVENTION

Accordingly, it is an object of the present invention to provide for implantation in a residual limb of an amputee, apparatus that has at least one biocompatible porous layer adapted for vascularization and stable sealable ingrowth by skin cells. A further object of the present invention is to provide for implantation in a residual limb of an amputee, apparatus that is not subject to failure by reason of infection. Another object of the present invention to provide for implantation in a residual limb of an amputee, apparatus that includes an uppermost biocompatible non-porous elastomer layer having a multiplicity of perforations. Still another object of the present invention is to provide for implantation in a residual limb of an amputee, apparatus that includes a biocompatible titanium mesh adapted for sealable cellular ingrowth. Even still a further object of the present invention is to provide for implantation in a residual limb of an amputee, apparatus that does not have high failure rates initially. Yet still a further object of this invention is to provide methods and apparatus that are suitable for use with a variety of polymeric materials. Even a further object of this invention is to provide apparatus for implantation in a residual limb of an amputee that provides enhanced osseoperception. Yet even an additional object of this invention is to provide an article of manufacture for packaging the apparatus of the invention. Even still an additional object of this invention is to provide a device capable of delivering an antimicrobial formulation to the wound.

These and other objects are accomplished by the parts, constructions, arrangements, combinations and subcombinations comprising the present invention, the nature of which is set forth in the following general statement, and preferred embodiments of which—illustrative of the best modes in which applicant has contemplated applying the principles—are set forth in the following description and illustrated in the accompanying drawings, and are particularly and distinctly pointed out and set forth in the appended claims forming a part hereof.

### BRIEF EXPLANATION OF THE DRAWINGS

The foregoing and other objects and advantages of the invention will be appreciated more fully from the following further description thereof, with reference to the accompanying drawings in which like parts are given like reference numerals and wherein:

FIG. 1 is a schematic rendering of an enlarged elevational view of a transcutaneous implant in accordance with the present invention.

FIG. 2 is a schematic rendering of an enlarged cross sectional view of the skin of a human patient.

FIG. 3 is a schematic rendering of an enlarged cross sectional view of a transcutaneous implant having a titanium bone implant post welded to a single biocompatible porous titanium mesh layer in accordance with the present invention.

FIG. 4 is a schematic rendering of an enlarged cross sectional view of a transcutaneous implant having one biocompatible porous layer and an uppermost biocompatible substantially non-porous elastomer layer having a multiplicity of perforations in accordance with the present invention.

FIG. 5 is a schematic rendering of an enlarged cross sectional view of a transcutaneous implant having two biocompatible porous layers and an uppermost biocompatible substantially non-porous elastomer layer having a multiplicity of perforations in accordance with the present invention.

FIG. 6 is a schematic rendering of an enlarged cross sectional view of a transcutaneous implant having three biocompatible porous layers and an uppermost biocompatible substantially non-porous elastomer layer having a multiplicity of perforations in accordance with the present invention.

FIG. 7 is a diagrammatic view of an article of manufacture, comprising packaging material, a transcutaneous implant, a label, and a container of antimicrobial formulation in accordance with the present invention.

### DETAILED DESCRIPTION OF THE PREFERRED EMBODIMENTS

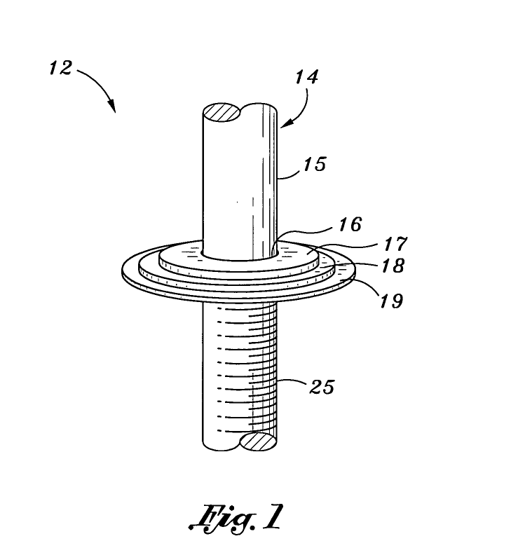

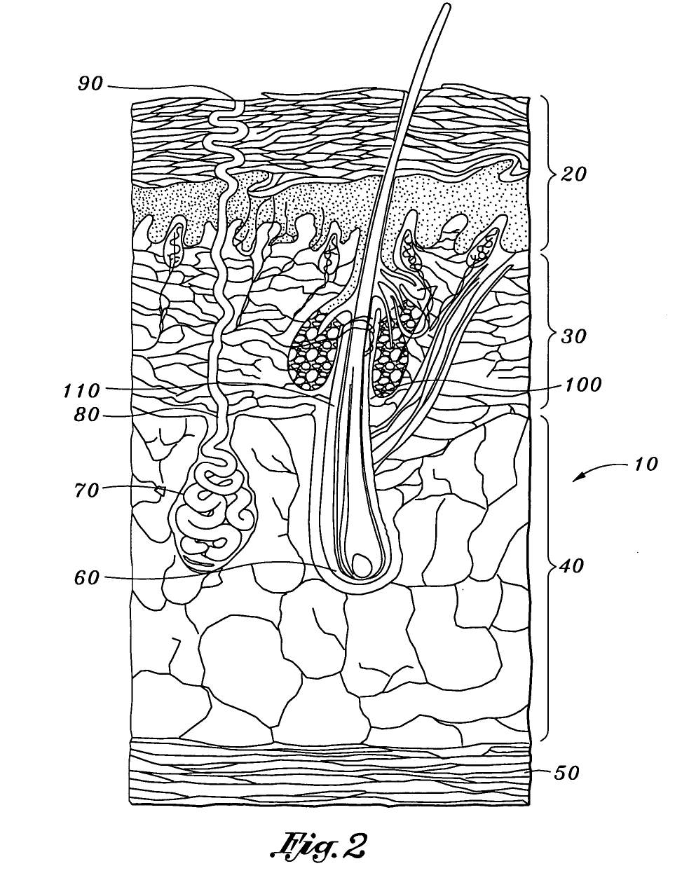

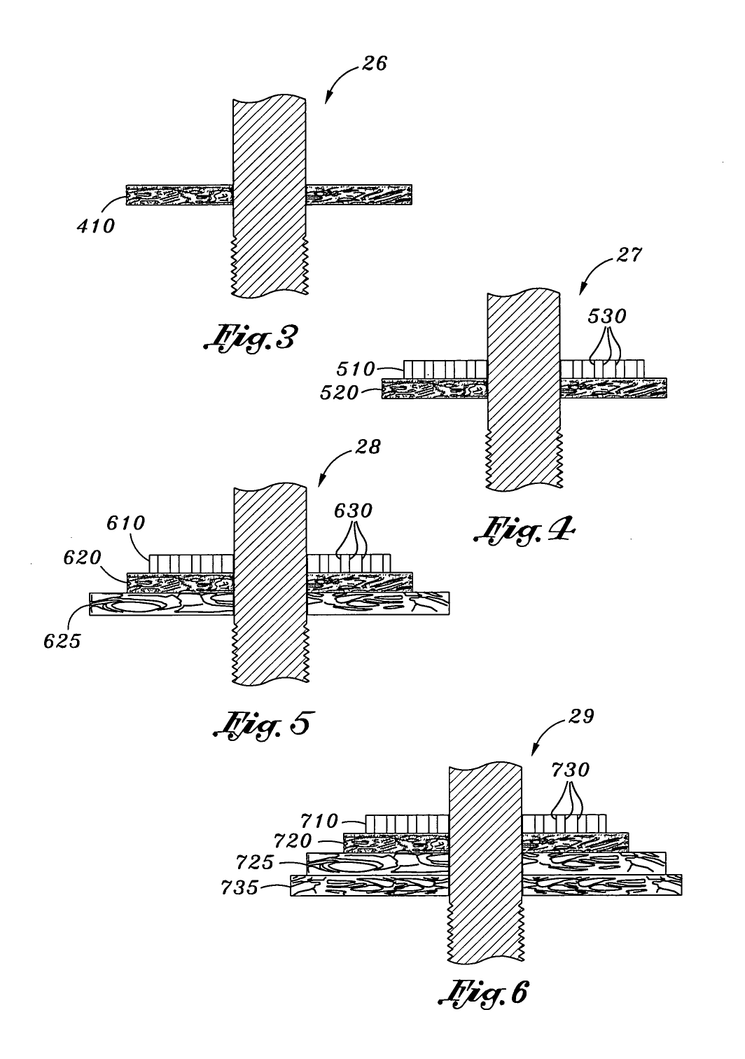

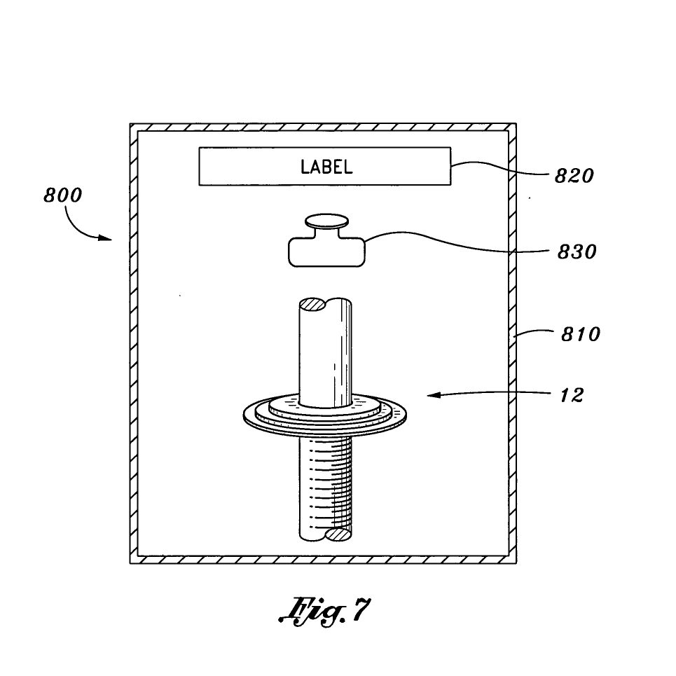

With reference to FIG. 1 a schematic rendering of a laminar transcutaneous implant in accordance with the present invention is shown and generally indicated at 12. An implant post generally indicated at 14 comprises an implantation segment 25 adapted for implantation in a bone within a residual limb and an attachment segment 15 adapted for attachment to a prosthesis for an amputee. A transcutaneous segment 16 is attached to a layer 17, a layer 18, and a layer 19 of implant 12. With reference to FIG. 2 a schematic rendering of an enlarged cross sectional view of normal human skin is shown and generally indicated at 10. The epidermis 20 overlies the dermis 30. Dermis 30 overlies the subcutaneous connective tissue 40. Underlying tissue 40 is the muscle layer 50. A hair papilla 60 and a sweat gland 70 are located in tissue 40. Gland 70 extends through a sweat gland duct 80 and exits epidermis 20 through a sweat gland pore 90. Sebaceous glands 100 lubricate hair follicle 110. With reference to FIG. 3, a schematic rendering of an enlarged cross sectional view is shown of a transcutaneous implant in accordance with the present invention having a titanium bone implant post generally indicated at 26 welded to a single biocompatible porous titanium mesh layer 410. With reference to FIG. 4 a schematic rendering of an enlarged cross sectional view of a transcutaneous implant having a biocompatible bone implant post generally indicated at 27 in accordance with the present invention is shown. Attached to post 27 is a flexible, porous biocompatible membrane 520 adapted for epidermal ingrowth to which a non-porous thermoplastic biocompatible elastomer 510, perforated by a multiplicity of laser induced ablations 530, is substantially non-delaminably bonded by known methods, for example by thermal bonding. With reference to FIG. 5 a schematic rendering of an enlarged cross sectional view of a transcutaneous implant having a biocompatible bone implant post generally indicated at 28 in accordance with the present invention is shown. Attached to post 28 is a flexible, porous biocompatible fluoropolymer membrane 625 adapted for dermal ingrowth to which a flexible, porous biocompatible membrane 620 adapted for epidermal ingrowth is substantially non-delaminably bonded by known methods, for example by thermal bonding. A flexible non-porous thermoplastic biocompatible elastomer 610, perforated by a multiplicity of laser induced ablations 630, is substantially non-delaminably bonded to membrane 620 by known methods, for example by thermal bonding. With reference to FIG. 6 a schematic rendering of an enlarged cross sectional view of a transcutaneous implant having a biocompatible bone implant post generally indicated at 29 in accordance with the present invention is shown. Attached to post 29 is a flexible, porous biocompatible fluoropolymer membrane 735 adapted for connective tissue ingrowth, to which a flexible, porous biocompatible fluoropolymer membrane 725 adapted for dermal ingrowth is substantially non-delaminably bonded by known methods, to which a flexible, porous biocompatible membrane 720 adapted for epidermal ingrowth is substantially non-delaminably bonded by known methods, for example by thermal bonding. A flexible non-porous thermoplastic biocompatible elastomer 710, perforated by a multiplicity of laser induced ablations 730, is substantially non-delaminably bonded to membrane 720 by known methods, for example by thermal bonding. With reference to FIG. 7 a diagrammatic view of an article of manufacture generally indicated at 800, comprising a packaging material 810, transcutaneous implant 12, a label 820 and a container 830 of a pharmaceutically acceptable topical antimicrobial formulation is shown.

A crucially important aspect of my invention is the interaction between the living dermis and/or epidermis of the patient and the porous membranes of the invention. Thus, in order to provide a laminar skin-bone fixation transcutaneous implant that can remain in place for an extended period, it is necessary that the device of the invention be sealably integrated with living tissue. This requirement—that of a tight seal that is impassable by infectious organisms—is met by ingrowth of skin cells into pores of porous biocompatible membranes, for example 720 or 725 of FIG. 6. The importance of the specific surface (cm²/g) as a function of pore size in this connection has been noted by Yannas et al. (Yannas, I. V., Lee, E., Orgill, D. P., Skrabut, E. M. and Murphy, G. F. (1989) Synthesis and characterization of a model extracellular matrix that induces partial regeneration of adult mammalian skin. Proc Natl Acad Sci USA, 86, 933-7). In my opinion, it is reasonable to believe that the specific limits on the mean pore diameter that govern the sealability of the membrane suggest that an ingrowth of tissue into the pores is required for sealability to be achieved, and this factor is incorporated in my invention.

Thus, my invention comprises a laminar skin-bone fixation transcutaneous implant adapted for implantation in a residual limb of an amputee comprising, in combination, a biocompatible bone implant post having a first segment adapted for implantation in a bone within the residual limb; a transcutaneous segment attached to each layer of the implant; a third segment adapted for attachment to a prosthesis for the amputee; and, at least one biocompatible substantially flexible porous layer adapted for cellular ingrowth attached to adjacent layers and to the transcutaneous segment. These biocompatible substantially flexible porous layers are adapted for stable sealable ingrowth by skin cells by virtue of their pore size, thickness, and chemical structure as explained in detail herein. The biocompatible substantially flexible porous layers may be fabricated from such materials as the elastomers explained in detail below, or from titanium mesh as explained in detail below. The implant may further comprise an uppermost biocompatible substantially flexible substantially non-porous elastomer layer having a multiplicity of perforations, wherein the uppermost biocompatible substantially non-porous elastomer layer is attached to adjacent layers and to the transcutaneous segment. The biocompatible bone implant post may be made from a metal such as titanium, titanium alloys, cobalt-chrome alloys, and stainless steel. One or more of the biocompatible substantially flexible porous layers may be a titanium mesh layer attached to the transcutaneous segment by welding. The titanium mesh layer may have a porosity of about 38-90% and an average pore diameter of about 30-400μ. The biocompatible substantially flexible porous layer may have an average pore diameter of about 0.5μ to about 400μ. The perforations in the substantially non-porous elastomer layer may have a diameter between about 0.2μ and about 10μ and the layer may have a thickness between about 25μ and about 1000μ. The perforations in this layer are advantageously produced using a laser. The substantially non-porous elastomer layer may be fabricated from vinylidene polymer plastics, polyethylene, polypropylene, polyesters, polyamides, polyethylene terephthalate, high density polyethylene, irradiated polyethylene, polycarbonates, polyurethanes, polyvinyl chloride, polyester copolymers, polyolefin copolymers, FEP, PFA (perfluoroalkoxy), PPS, PVDF (polyvinylidene fluoride), PEEK, PS/PES, PCTFE, or PTFE. Particularly useful for this purpose is FEP. The implant may comprise a first biocompatible substantially flexible porous polymer layer and a second biocompatible substantially flexible porous polymer layer situated between the first biocompatible substantially flexible porous polymer layer and the bone. The implant may include a biocompatible substantially flexible porous polymer layer comprising one or more porous fluorocarbon polymer layers made from PTFE, ePTFE, FEP, PFA, PVDF, PCTFE, or ETFE. The biocompatible substantially flexible porous polymer layer may have a thickness between about 25μ and about 3000μ and may be saturated with a pharmaceutically acceptable topical antimicrobial formulation that includes one or more of the following substances: polymyxin B, neomycin, mupirocin, amphotericin B, nystatin, norfloxacin, and ciprofloxacin. The implant may include a third or fourth biocompatible substantially flexible porous polymer layer having pores that have an average diameter between about 50μ and about 500μ. This layer may have a thickness between about 200μ and about 3000μ and is made from an elastomer such as PTFE, ePTFE, FEP, PFA, PVDF, PCTFE, or ETFE and situated between the first biocompatible substantially flexible porous polymer layer and the second biocompatible substantially flexible porous polymer layer.

A method for the treatment of amputation comprises applying the implant of my invention in an amputee in need of such treatment, wherein the application is effective as part of a procedure to ameliorate one or more of the effects of the amputation. An article of manufacture comprises packaging material and the implant of my invention contained within the packaging material, wherein the implant is effective for application to an amputee, and the packaging material includes a label that indicates that the implant is effective for the application. The article of manufacture may further comprise a container of a pharmaceutically acceptable topical antimicrobial formulation wherein the formulation may include one or more of the following substances: polymyxin B, neomycin, mupirocin, amphotericin B, nystatin, norfloxacin, and ciprofloxacin.

Formation of Titanium Fiber Mesh Layers

Compacting a single titanium fiber into a die to achieve the desired porosity, followed by vacuum sintering forms titanium fiber mesh layers.

Welding Titanium Structures.

Biocompatible titanium and most titanium alloys are readily welded by a number of welding processes known in the art. The most common method of joining titanium is the gas tungsten—arc (GTAW) process and, secondarily, the gas metal—arc (GMAW) process. Others include electron beam, and more recently laser welding as well as solid state processes such as friction welding and diffusion bonding. Titanium and its alloys also can be joined by resistance welding and by brazing. Titanium mesh is attached to a titanium post by means of the above mentioned welding procedures.

Attaching Elastomer Membranes to Titanium

Using a laser beam to melt the elastomer into contact with the titanium forms the titanium-elastomer attachments required for my invention. Preferably, the titanium surface to be attached to the elastomer is first pitted with the aid of a laser to provide a roughened surface that facilitates the attachment.

Formation of Bilaminar ePTFE/FEP Films

Unlike unmodified (PTFE), which does not adhere to almost any other material, the production of porosity in ePTFE results in a material than can be bonded to other materials and to itself. This is true because bonding agents are able to penetrate a significant distance into the pore network, and, after hardening, they become locked in place. For example, a layer of FEP film can be contacted with an ePTFE film and the resulting combination can be heated in an oven at about 320° C. for about 5 minutes. This period of time is adequate to allow for melting of the FEP in an amount which, following removal of the assembly from the oven and cooling, results in a stable bilaminate film, wherein one surface is porous and the other surface is non-porous. In general, two elastomer membranes as discussed in the description of my invention can be bonded to each other to form a bilaminate structure using the techniques discussed in this paragraph.

Loading Of Porous Membranes With Therapeutic Substances

The void spaces of porous membranes are loaded with any of a variety of therapeutic substances including antimicrobial substances, antiinflammatory substances, growth modulators and the like for the control of infection, inflammation and other biological process that may be of importance in connection with the placement of the membrane on the amputation residual limb.

Thus it will be appreciated that the invention provides a new and improved laminar skin-bone fixation transcutaneous implant. It should be understood, however, that the foregoing description of the invention is intended merely to be illustrative thereof and that other modifications in embodiments may be apparent to those skilled in the art without departing from its spirit. On this basis, the instant invention should be recognized as constituting progress in science and the useful arts, and as solving the problems in orthopedics and medicine enumerated above. In the foregoing description, certain terms have been used for brevity, clearness and understanding, but no unnecessary limitation is to be implied therefrom beyond the requirements of the prior art, because such words are used for descriptive purposes herein and are intended to be broadly construed.

Having described preferred embodiments of the invention with reference to the accompanying drawings, it is to be understood that the invention is not limited to those precise embodiments, and that the various changes and modifications may be effected therein by one skilled in the art without departing from the scope or spirit of the invention as defined in the appended claims. Thus, the scope of the invention should be determined by the appended claims and their legal equivalents, rather than by the examples given. All changes that come within the meaning and range of equivalency of the claims are to be embraced within their scope.

### Definitions

Unless defined otherwise, all technical and scientific terms used herein have the same meaning as is commonly understood by one of skill in the art to which this invention belongs. All patents and publications referred to herein are incorporated in their entirety by reference.

All abbreviations for fluorocarbon polymers used herein have the same meaning as is commonly understood by one of skill in the art to which this invention belongs. For example, PTFE refers to polytetrafluoroethylene, and ePTFE refers to expanded polytetrafluoroethylene. As further examples, FEP refers to poly(tetrafluoroethylene-co-hexafluoropropylene, PFA refers to perfluoroalkoxyalkene copolymer, PVDF refers to polyvinylidene fluoride, PCTFE refers to polychlorotrifluoroethylene, and ETFE refers to ethylene tetrafluoroethylene.

All terms for polymers used herein have the same meaning as is commonly understood by one of skill in the art to which this invention belongs. As an example, the terms “resin”, “polymer”, and “elastomer” may be used synonymously by one of skill in the art to which this invention belongs.

As used herein, “biocompatible” means not having toxic or injurious effects on biological function in a host.

As used herein, a laminar structure is a structure comprising at least one layer.

As used herein, a bilaminate structure is a structure comprising two layers.

As used herein, a trilaminate structure is a structure comprising three layers.

As used herein, a non-delaminable structure is a structure comprising at least two layers wherein the layers cannot be pulled apart or separated from each other without destroying the structural integrity of the individual layers.

As used herein, the terms “sealable” and “sealably” refer to a seal sufficiently tight to block the passage of infectious organisms.

As used herein, the average pore diameter in a sample of porous polymer is the average value, expressed in p, that is obtained using an electron microscope according to the method of Dagalakis et al., (Dagalakis, N., Flink, J., Stasikelis, P., Burke, J. F. and Yannas, I. V. (1980) Design of an artificial skin. Part Ill. Control of pore structure. J Biomed Mater Res, 14, 511-28).

As used herein, the skin is the membranous, protective covering of the human body consisting of epidermis and dermis.

As used herein, an amputee is a patient with a residual limb.

As used herein, a residual limb includes those parts remaining after amputation or avulsion of portions of an arm, a forearm, a finger, a thumb, a thigh, a calf of a leg, or a toe of a patient.

As used herein, a lowermost layer of a transcutaneous laminar implant is that layer that is closest to the bone being implanted; an uppermost layer is that layer that is furthest from the bone being implanted; and, an intermediate layer is situated between an uppermost layer and a lowermost layer.

As used herein, therapeutic substances include antimicrobial substances, antiinflammatory substances, growth modulators and the like for the control of infection, inflammation and other biological process that may be of importance in connection with the placement of the membrane on the amputation residual limb.

What is claimed is:

1. A laminar skin-bone fixation transcutaneous implant adapted for implantation in a residual limb of an amputee comprising, in combination:
a biocompatible bone implant post having a first segment adapted for implantation in a bone within said residual limb; a transcutaneous segment attached to each layer of said implant; and, a third segment adapted for attachment to a prosthesis for said amputee; and,
at least one biocompatible substantially flexible porous layer adapted for cellular ingrowth attached to adjacent layers and to said transcutaneous segment;
wherein said at least one biocompatible substantially flexible porous layer is adapted for stable sealable ingrowth by skin cells.

2. The implant of claim 1, further comprising an uppermost biocompatible substantially flexible substantially non-porous elastomer layer having a multiplicity of perforations, wherein said uppermost biocompatible substantially non-porous elastomer layer is attached to adjacent layers and to said transcutaneous segment.

3. The implant of claim 1, wherein said biocompatible bone implant post is made from a metal selected from the group consisting of titanium, titanium alloys, cobalt-chrome alloys, and stainless steel.

4. The implant of claim 3, wherein one or more of said at least one biocompatible substantially flexible porous layer adapted for cellular ingrowth is a titanium mesh layer attached to said transcutaneous segment by welding.

5. The implant of claim 4, wherein said one or more titanium mesh layer comprises a mesh having a porosity of about 38-90% and an average pore diameter of about 30-400μ.

6. The implant of claim 1, wherein one or more of said at least one biocompatible substantially flexible porous layer has an average pore diameter of at least about 0.5μ to about 40μ.

7. The implant of claim 1, wherein one or more of said at least one biocompatible substantially flexible porous layer has an average pore diameter of at least about 41μ to about 400μ.

8. The implant of claim 2, wherein said perforations in said substantially non-porous elastomer layer have a diameter between at least about 0.2μ and about 10μ.

9. The implant of claim 2, wherein said substantially non-porous elastomer layer has a thickness between at least about 25μ and about 1000μ.

10. The implant of claim 2, wherein said substantially non-porous elastomer layer is selected from the group consisting of vinylidene polymer plastics, polyethylene, polypropylene, polyesters, polyamides, polyethylene terephthalate, high density polyethylene, irradiated polyethylene, polycarbonates, polyurethanes, polyvinyl chloride, polyester copolymers, polyolefin copolymers, FEP, PFA (perfluoroalkoxy), PPS, PVDF (polyvinylidene fluoride), PEEK, PS/PES, PCTFE, and PTFE.

11. The implant of claim 1, wherein said at least one biocompatible substantially flexible porous layer comprises a first biocompatible substantially flexible porous polymer layer and a second biocompatible substantially flexible porous polymer layer situated between said first biocompatible substantially flexible porous polymer layer and said bone.

12. The implant of claim 1, wherein said at least one biocompatible substantially flexible porous polymer layer comprises one or more porous fluorocarbon polymer layers each individually selected from the group consisting of PTFE, ePTFE, FEP, PFA, PVDF, PCTFE, and ETFE.

13. The implant of claim 1, wherein one or more of said at least one biocompatible substantially flexible porous polymer layer has a thickness between at least about 25μ and about 3000μ.

14. The implant of claim 1, wherein one or more of said at least one biocompatible substantially flexible porous polymer layer is saturated with a pharmaceutically acceptable topical therapeutic formulation.

15. The implant of claim 14, wherein said formulation includes one or more of the following substances: polymyxin B, neomycin, mupirocin, amphotericin B, nystatin, norfloxacin, and ciprofloxacin.

16. The implant of claim 11, further comprising a third biocompatible substantially flexible porous polymer layer situated between said first biocompatible substantially flexible porous polymer layer and said second biocompatible substantially flexible porous polymer layer.

17. The implant of claim 16, wherein said third biocompatible substantially flexible porous polymer layer is a fluorocarbon polymer layer selected from the group consisting of porous PTFE, ePTFE, FEP, PFA, PVDF, PCTFE, and ETFE.

18. The implant of claim 16, wherein said third biocompatible substantially flexible porous polymer layer has a thickness between at least about 200μ and about 3000μ.

19. The implant of claim 18, wherein the pores in said third biocompatible substantially flexible porous polymer layer have an average diameter between at least about 50μ and about 500μ.

20. A method for the treatment of amputation, comprising applying the implant of claim 1 in an amputee in need of such treatment, wherein said application is effective as part of a procedure to ameliorate one or more of the effects of said amputation.

21. An article of manufacture, comprising packaging material and the implant of claim 1 contained within the packaging material, wherein said implant is effective for application to an amputee, and the packaging material includes a label that indicates that said implant is effective for said application.

22. The article of manufacture of claim 21, further comprising a container of one or more pharmaceutically acceptable therapeutic substances.

23. The article of manufacture of claim 22, wherein said substances comprise one or more of the following antimicrobial substances: polymyxin B, neomycin, mupirocin, amphotericin B, nystatin, norfloxacin, and ciprofloxacin.

24. A laminar skin-bone fixation transcutaneous implant adapted for implantation in a residual limb of an amputee comprising, in combination:
a biocompatible bone implant post made from a metal selected from the group consisting of titanium, titanium alloys, cobalt-chrome alloys, and stainless steel, said implant having a first segment adapted for implantation in a bone within said residual limb; a transcutaneous segment attached to each layer of said implant; and, a third segment adapted for attachment to a prosthesis for said amputee; and,
at least one biocompatible substantially flexible porous layer adapted for cellular ingrowth attached to adjacent layers and to said transcutaneous segment;
wherein one or more of said at least one biocompatible substantially flexible porous layer adapted for cellular ingrowth is a titanium mesh layer attached to said transcutaneous segment by welding; and,
wherein said at least one biocompatible substantially flexible porous layer is adapted for stable sealable ingrowth by skin cells.

25. The implant of claim 24, wherein said one or more titanium mesh layers comprises a mesh having a thickness of about 0.5-1.5 cm., a porosity of about 38-90%, and, an average pore diameter of about 30-400μ.

26. The implant of claim 24, further comprising an uppermost biocompatible substantially flexible substantially non-porous elastomer layer having a multiplicity of perforations, wherein said uppermost biocompatible substantially non-porous elastomer layer is attached to an adjacent layer and to said transcutaneous segment.

27. A laminar skin-bone fixation transcutaneous implant adapted for implantation in a residual limb of an amputee comprising, in combination:
a biocompatible bone implant post made from a metal selected from the group consisting of titanium, titanium alloys, cobalt-chrome alloys, and stainless steel, said implant having a first segment adapted for implantation in a bone within said residual limb; a transcutaneous segment attached to each layer of said implant; and, a third segment adapted for attachment to a prosthesis for said amputee; and,
two biocompatible substantially flexible porous layers adapted for cellular ingrowth attached to each other and to said transcutaneous segment;
wherein said two biocompatible substantially flexible porous layers are adapted for stable sealable ingrowth by skin cells.

28. The implant of claim 27, further comprising an uppermost biocompatible substantially flexible substantially non-porous elastomer layer having a multiplicity of perforations, wherein said uppermost biocompatible substantially non-porous elastomer layer is attached to an adjacent layer and to said transcutaneous segment.

29. A laminar skin-bone fixation transcutaneous implant adapted for implantation in a residual limb of an amputee comprising, in combination:
a biocompatible bone implant post made from a metal selected from the group consisting of titanium, titanium alloys, cobalt-chrome alloys, and stainless steel, said implant having a first segment adapted for implantation in a bone within said residual limb; a transcutaneous segment attached to each layer of said implant; and, a third segment adapted for attachment to a prosthesis for said amputee; and,
three biocompatible substantially flexible porous layers adapted for cellular ingrowth attached to each other and to said transcutaneous segment;
wherein said three biocompatible substantially flexible porous layers are adapted for stable sealable ingrowth by skin cells.

30. The implant of claim 29, further comprising an uppermost biocompatible substantially flexible substantially non-porous elastomer layer having a multiplicity of perforations, wherein said uppermost biocompatible substantially non-porous elastomer layer is attached to an adjacent layer and to said transcutaneous segment.
